(19) United States
(12) Patent Application Publication
(10) Pub. No.: US 2021/0290851 A1
(43) Pub. Date: Sep. 23, 2021

(54) Ergonomic Fine-Precision Injection And Aspiration Safety Device For Aesthetic Injections

(71) Applicants: Jacob Richard Thompson, Castle Rock, CO (US); Sadie Marie Sellam, Castle Rock, CO (US)

(72) Inventors: Jacob Richard Thompson, Castle Rock, CO (US); Sadie Marie Sellam, Castle Rock, CO (US)

(21) Appl. No.: 17/207,245

(22) Filed: Mar. 19, 2021

Related U.S. Application Data
(60) Provisional application No. 62/992,454, filed on Mar. 20, 2020.

(51) Int. Cl. A61M 5/31; A61M 5/315

(52) U.S. Cl. CPC A61M 5/3148; A61M 2202/048; A61M 5/315; A61M 5/3137

(57) ABSTRACT

The present invention comprises a novel fine-precision injection device comprising a motor-driven system which couples with the plunger of generic, disposable syringes and an ergonomic grip which interlocks with the main body of the drive system. The grip comprises an object shaped with depressions matching the fingers of a hand and has integrated buttons within the finger depressions which allow actuation of the controls intended by the device and may have a snap-fit interlocking mechanism. The drive system may comprise a forward-located motor which drives gears and a threaded leadscrew to force a carriage back and forth. The carriage is coupled with the plunger of a disposable syringe while the body of the syringe is loaded into the main body of the drive system. The main body may comprise electronics and appropriate buttons to allow configuration and operation of the device.

### CROSS-REFERENCES TO RELATED APPLICATIONS

This application claims the benefit of U.S. Provisional Application Ser. No. 62/992,454 filed on Mar. 20, 2020 and titled “ERGONOMIC FINE-PRECISION INJECTION AND ASPIRATION SAFETY DEVICE FOR AESTHETIC INJECTIONS” which is incorporated herein by reference in its entirety for all that is taught and disclosed therein.

### BACKGROUND

This application relates to the field of aesthetic injection devices used for injection of botulinum toxin or dermal fillers.

### SUMMARY OF THE INVENTION

The present invention comprises a novel fine-precision injection device that supports ergonomic control and aspiration of fluid consisting of a motor-driven system for capturing the plunger of generic, disposable syringes and a press-fit, locking grip that holds the syringe and main body of the drive system. The grip is shaped with depressions which support the fingers in a manner that reduces fatigue and strain of the fingers, wrist and arm and has integrated buttons within the finger depressions which allow actuation of the controls for the tasks of injection, injection volume control, and aspiration. The drive system features a plunger-capturing carriage with the ability to accept generic disposable syringes, a forward-located motor to aid in allocating the weight toward the front of the device to reduce weight-related strain, and the ability to both depress and retract the syringe plunger for injection and aspiration.

The depression and retraction of the syringe plunger is handled automatically upon actuation with the appropriate buttons to eject the volume desired from the syringe and into the patient. The desired ejection volume is controlled by the user with a separate actuator, which toggles various modes adjusting the ejection volume. The ejection volume is calculated by a measurement of the inner diameter (ID) of the chosen disposable syringe. An integrated Light-Emitting Diode (LED) screen offers awareness over the status of the current injection session and collocated buttons allow configuration of the device's parameters and plunger-capturing carriage's position.

The proposed value of this invention is that current inventions and products oriented toward aesthetic injections do not sufficiently allow for the injection administrator to validate whether or not an injection would cause medical fluid to enter the bloodstream. Aspiration, a technique to detect whether or not a needle is present in the bloodstream by retracting the syringe plunger and checking for the presence of blood within the syringe, is difficult to achieve with current mechanisms. With a regular syringe, aspiration is accomplished by placing the needle and attempting to hold the entire syringe steady while bringing one hand far enough back to pull back on the syringe plunger. This is often difficult given that three simultaneous tasks are generally required: pulling skin taut, inserting and holding the needle steady, and pushing or pulling the plunger. Other devices available do not address this need.

The field of aesthetic injections often involves injection of either botulinum toxin or dermal fillers. Botulinum toxin is dangerous if injected into the bloodstream and can cause blindness. Dermal fillers are able to cause skin necrosis, or dead skin cells, when injected directly into the bloodstream. Aspiration is the most reliable mechanism to detect whether or not the needle is placed into a blood vessel.

Most other devices focus entirely on injection and its accuracy but do not address this aspect of aspiration. Similarly, existing devices do not account for the ergonomic aspects of fine-precision injections while enabling the previously mentioned simultaneous tasks. The available devices lack the ability to finely control the needle by holding the end closest to the needle or, if they do then they lack any ability to aspirate for patient safety. Additionally, available devices are often coupled with very specific syringe types that are a requirement for use. This creates vendor lock-in and raises supply chain risks associated with buying any existing device.

Finally, many injection establishments are often governed by a medical director, who simply accredits an establishment and assumes the risk involved in administering injections of the associated medical fluids. Without a direct presence of the director to oversee operations, there can be instances of poorly trained or unsupervised personnel who are faced with a difficulty in effectively executing aspiration with each injection at every skin location within an allotted treatment timeframe. The proposed invention addresses all of the mentioned deficiencies.

Other applications may be appropriate for this invention as well, such as scalp injections for the purpose of reversing or slowing hair loss. Corticosteroid injections as well as platelet-rich-plasma injections are prime candidates for precision injection procedures whose goal is not to inject aesthetic medical fluids into blood vessels, but rather require a distributed dispersal of fluid to many areas of the skin in a single treatment.

### BRIEF DESCRIPTION OF THE DRAWINGS

FIG. 1 shows the overall embodiment of the front side of the present invention.

FIG. 2 shows the overall embodiment of the rear side of the present invention.

FIG. 3 shows the overall embodiment of the present invention from a top-front perspective.

FIG. 4 shows the overall embodiment of the present invention from a top-rear perspective.

FIG. 5 shows the overall embodiment of the present invention from a bottom-front perspective.

FIG. 6 shows the overall embodiment of the present invention from a bottom-rear perspective.

FIG. 7 shows an embodiment of the present invention as if it were being decoupled into two constituent parts, a handgrip and the central housing, from an orthogonal front view.

FIG. 8 shows an embodiment of the present invention as if it were being decoupled into three constituent parts, a handgrip, central housing and disposable syringe, from a top-rear perspective.

FIG. 9 shows an embodiment of the handgrip, motor housing, and syringe body of the present invention in an exploded view from a top-front perspective. The internal motor is not shown within the motor housing and the plunger is not present within the syringe body.

FIG. 10 shows an embodiment of the central housing with the internal motor and gear exposed in an exploded view from a top-rear perspective. The top and rear panels of the central housing are not shown to expose the internal components.

FIG. 11 shows an embodiment of more of the internal components of the central housing in an exploded view from a top-rear perspective. The drive system of the central housing is depicted. The rear and top panels of the central housing are not shown in addition to the motor and its cover.

FIG. 12 shows the same components as FIG. 11 but depicts them from the opposite side.

FIG. 13 shows an embodiment of the core drive system within the central housing from a top-down orthogonal view. All outer panels and the guide rail are not shown. The internal gears, drive screw, and carriage are depicted.

FIG. 14 shows the same components as FIG. 13 but depicts them from a top-rear perspective.

FIG. 15 shows an embodiment of electronic components in a top-front exploded view. The internal electronics and surface actuators are depicted.

FIG. 16 shows the same components as FIG. 15 but depicts them from a top-rear perspective.

FIGS. 17A and 17B show a procedural flow diagram of performing a treatment with the proposed embodiment of the present invention.

FIG. 18 shows an embodiment of the LED screen and potential values in a specific mode of operation.

FIG. 19 shows an embodiment of the LED screen and potential values in a specific mode of operation different from that of FIG. 18.

To assist in the understanding of the present disclosure the following list of components and associated numbering found in the drawings is provided herein:

Table of Components

| Component | # |
|---|---|
| Device | 10 |
| Syringe | 18 |
| Syringe Body | 20 |
| Syringe Needle | 22 |
| Syringe Plunger | 24 |
| Hand Grip | 26 |
| Hand Grip-Index | 28 |
| Finger Depression | |
| Hand Grip-Ring and Pinky | 30 |
| Finger Depressions | |
| Hand Grip-Thumb Depression | 32 |
| Hand Grip-Middle Finger | 34 |
| Depression | |
| Hand Grip-Palm Ridge | 36 |
| Hand Grip-Index | 38 |
| Finger Actuator | |
| Hand Grip-Middle | 40 |
| Finger Actuator | |
| Hand Grip-Thumb Actuator | 42 |
| Hand Grip-Flat Base | 44 |
| Main Body | 45 |
| Main Body-Motor Cover | 46 |
| Main Body-Central Housing | 48 |
| Main Body-Battery Cover | 50 |
| Battery Pack | 52 |
| Main Body-Rear Panel | 54 |
| Main Body-End Cover | 56 |
| Main Body-Central Column | 58 |
| Main Body-Front Panel | 60 |
| LED Screen | 62 |
| Main Body-Top Panel | 64 |
| Main Body-Bottom Panel | 66 |
| Hand Grip-Main Body Cutout | 68 |
| Hand Grip-Actuator Wire Pins | 70 |
| Main Body-Syringe Cavity | 72 |
| Syringe Plunger-Flange | 74 |
| Drive Gear | 76 |
| Motor | 78 |
| Plunger Carriage | 80 |
| Plunger Carriage Flange Cavity | 82 |
| Screw Nut | 84 |
| Guide Rail Carriage | 86 |
| Guide Rail | 88 |
| Screw Gear Cover | 90 |
| Drive Screw | 92 |
| Screw Gear | 94 |
| Motor Gear Cover | 96 |
| Wire Cavity | 98 |
| Battery Plug | 100 |
| Electronic Control Unit | 102 |
| Up Button | 104 |
| Configuration Button | 106 |
| Down Button | 108 |
| Speed Button | 110 |
| Syringe Button | 112 |
| Reset Button | 114 |
| Multiplier Button | 116 |

### DETAILED DESCRIPTION

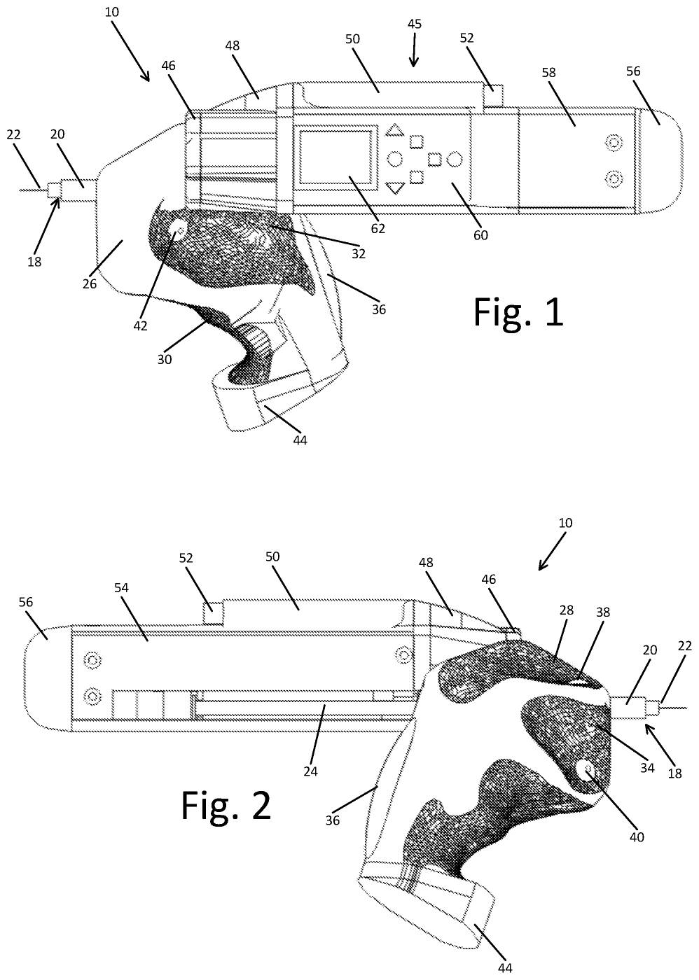

Referring now to the Figures, in which like reference numerals refer to structurally and/or functionally similar elements thereof, FIG. 1 shows a frontal schematic of a fully assembled embodiment of an ergonomic fine-precision injection and aspiration safety device 10 of the present invention (hereinafter simply referred to as device 10. Referring now to FIG. 1, a full embodiment of the present invention is displayed. The primary components consist of an ergonomic hand grip 26 and a main body 45. The ergonomic hand grip 26 shows ring and pinky finger depressions 30, a thumb depression 32, a palm ridge 36, a thumb actuator 42, and a flat base 44. Together with other component features, the ergonomic hand grip 26 supports comfortable use of the invention and eliminates strain and fatigue related to injection services. The thumb actuator 42 controls the amount of injection applied.

The ergonomic hand grip 26 interlocks with the central housing 48. The central housing 48 conjoins many parts of the main body 45. The motor cover 46, battery cover 50, battery pack 52, end cover 56, central column 58, and front panel 60 with LED Screen 62 are all fixed to the central housing 48. A syringe 18 has a disposable syringe body 20 and syringe needle 22 and a syringe plunger 24 (see FIG. 8) are displayed for reference though they are not directly part of the present invention.

FIG. 2 shows a rear schematic of a fully assembled embodiment of device 10 of the present invention. Referring now to FIG. 2, a full embodiment of the present invention is displayed. In this view, the index finger depression 28 and the index finger actuator 38 are visible in addition to the middle finger depression 34 and the middle finger actuator 40. The rear panel 54 is also visible. The syringe body 20 and syringe needle 22 are again shown for reference. In addition, the syringe plunger 24 is shown in FIG. 8. The index finger actuator 38 activates injection, pushing the syringe plunger 24 forward. The middle finger actuator activates aspiration, drawing the syringe plunger 24 in the reverse direction.

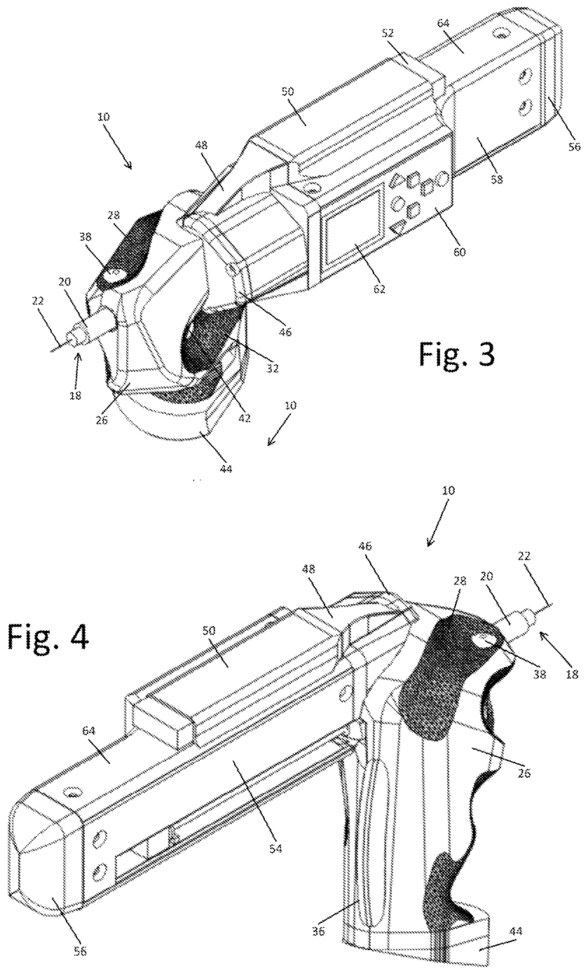

FIG. 3 shows a perspective schematic of a full embodiment of device 10 of the present invention. Referring now to FIG. 3, a component newly visible in this figure is the top panel 64.

FIG. 4 shows a perspective schematic of a full embodiment of device 10 of the present invention. Referring now to FIG. 4, no new components are introduced and the view is provided for completeness.

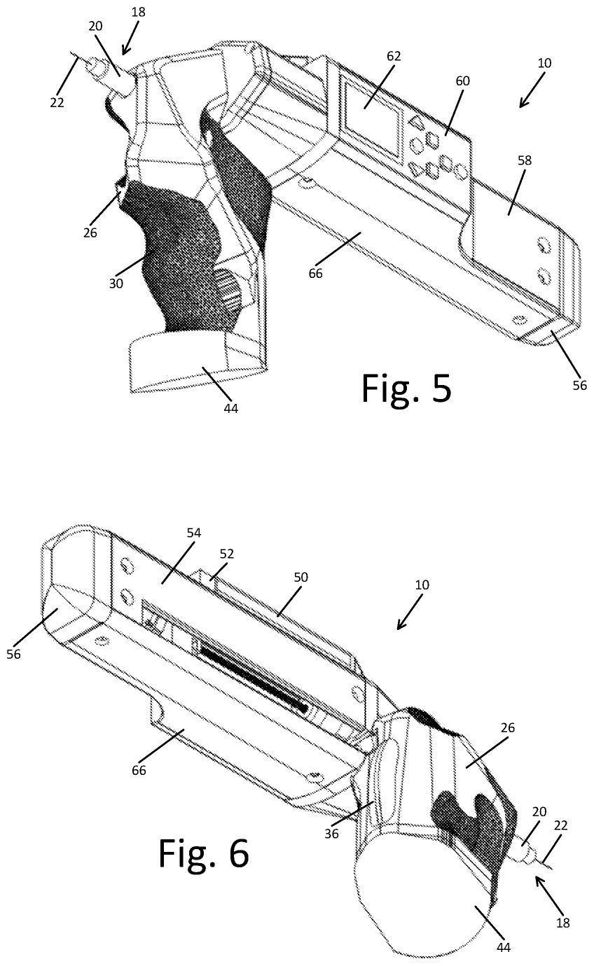

FIG. 5 shows a perspective schematic of a full embodiment of device 10 of the present invention. Referring now to FIG. 5, a component newly visible in this figure is the bottom panel 66.

FIG. 6 shows a perspective schematic of a full embodiment of device 10 of the present invention. Referring now to FIG. 6, no new components are introduced and the view is provided for completeness.

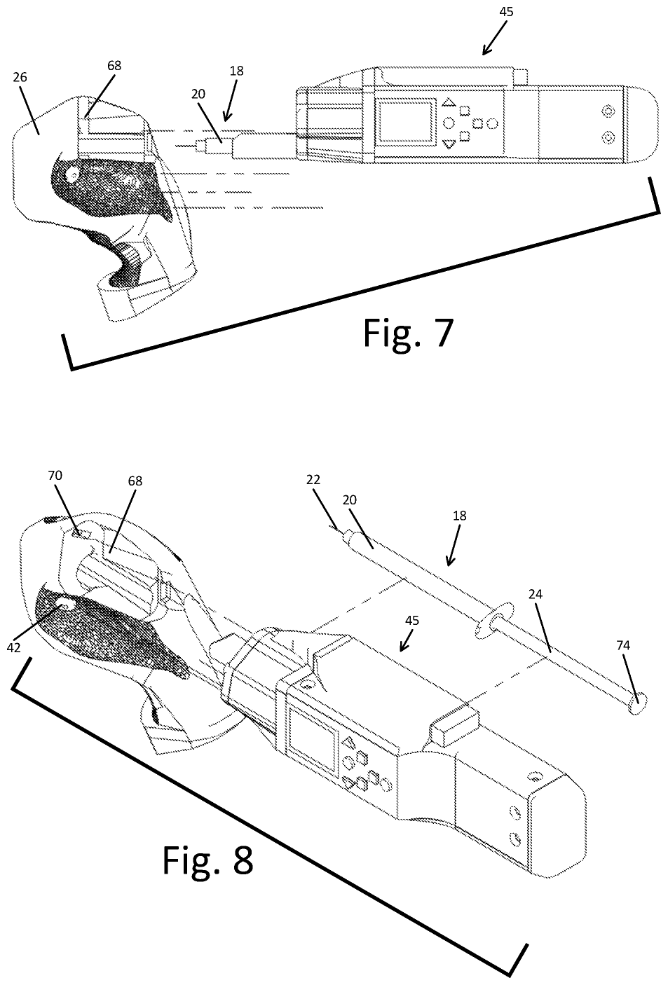

FIG. 7 shows an exploded schematic of the two primary components of the present invention. Referring now to FIG. 7, the main body 45 and syringe body 20 are decoupled from the ergonomic hand grip 26. This exposes the main body cutout 68 of the ergonomic hand grip 26 into which the main body 45 fits.

FIG. 8 shows an exploded schematic of the two primary components of the present invention in addition to a disposable syringe. Referring now to FIG. 8, the actuator wire pins 70 are visible, which connect correspondingly to the main body 45 and electrically enable the ergonomic hand grip thumb actuator 42, index finger actuator (not shown), and middle finger actuator (not shown). The disposable syringe is characterized by the syringe body 20, syringe needle 22, syringe plunger 24, and syringe plunger flange 74.

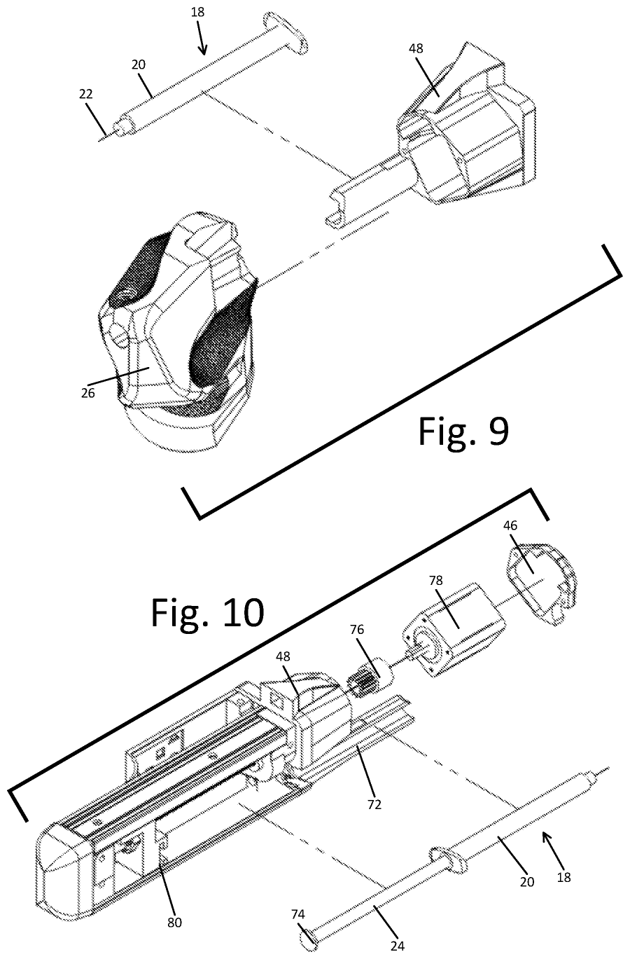

FIG. 9 shows an exploded view of the present invention. Referring now to FIG. 9, the central housing 48, ergonomic hand grip 26, and syringe body 20 are shown in greater detail. The central housing 48 is shown without the motor cover (not shown) or motor (not shown). The syringe body 20 fits into the central housing 48 prior to the central housing 48 fitting into the ergonomic hand grip 26.

FIG. 10 shows an exploded view of the main body 45 and its subcomponents.

Referring now to FIG. 10, the drive gear 76 attaches to the motor 78 and both are inserted into the central housing 48, secured by the motor cover 46. The syringe body 20 fits into the syringe cavity 72 of the central housing 48. The plunger carriage 80, which captures the syringe plunger flange 74 is also shown.

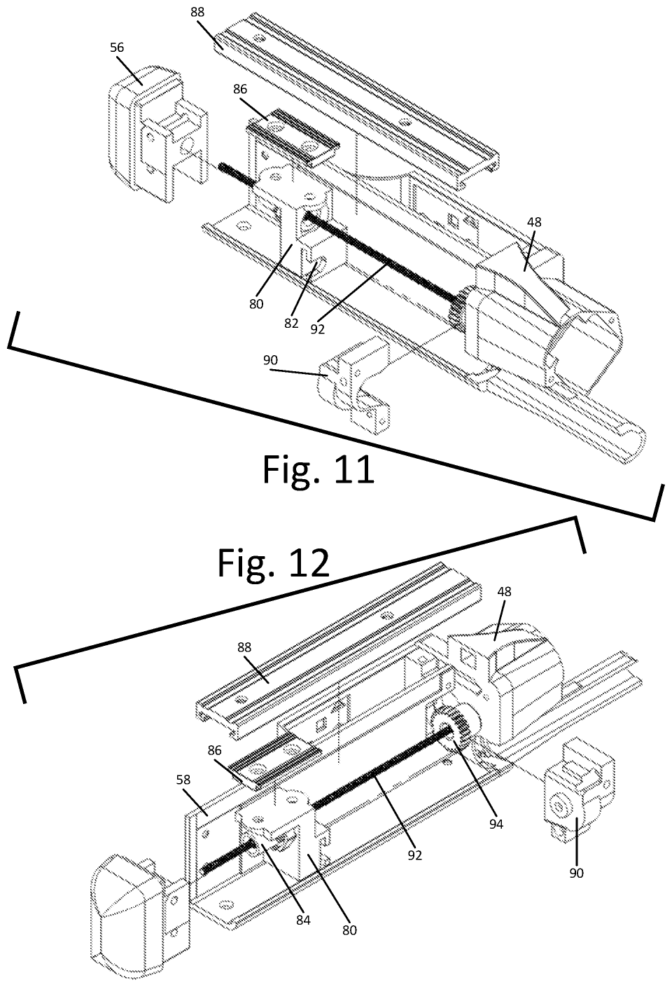

FIG. 11 shows an internal exploded view of the main body. Referring now to FIG. 11, the drive screw 92 can be seen passing through the plunger carriage 80 and into the end cover 56. The plunger carriage 80 has a plunger carriage flange cavity 82 which captures and controls the syringe plunger flange 74 (not shown) to enable injection and aspiration operations. The plunger carriage 80 is affixed to a guide rail carriage 86, which slides within a guide rail 88 to stabilize it.

FIG. 12 shows the same exploded arrangement of components as FIG. 11 but from an alternative viewpoint. Referring now to FIG. 12, the screw nut 84 is now visible. The screw nut 84 threads onto the drive screw 92 and is affixed to the plunger carriage 80. When the drive screw 92 is rotated by the screw gear 94, the screw nut 84 is driven forward or backward, subsequently moving the affixed plunger carriage 80 along the track of the guide rail 88 via the guide rail carriage 86. The screw gear 94 is covered by the screw gear cover 90. The central column 58 is also seen from an internal perspective.

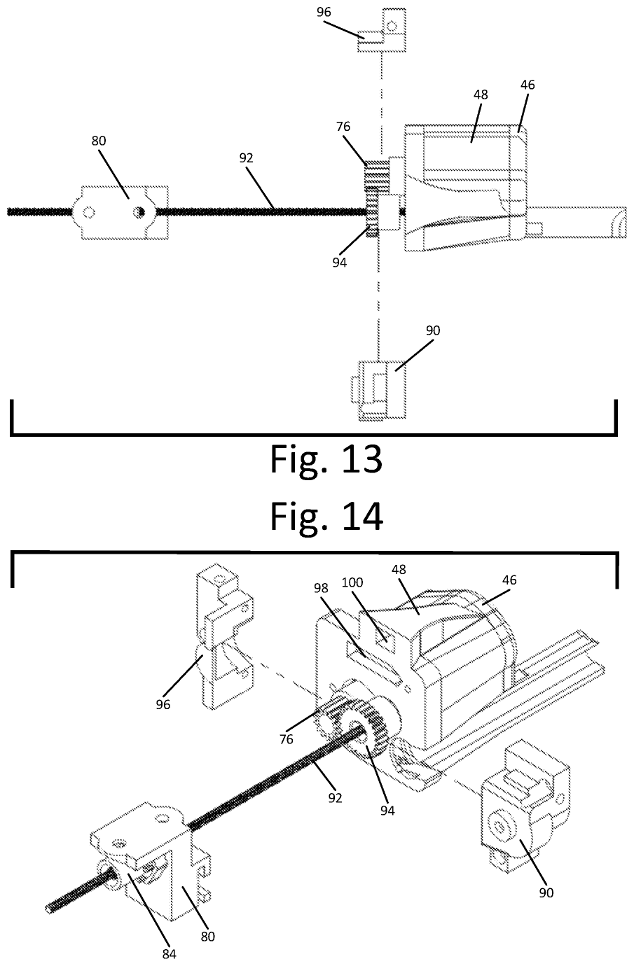

FIG. 13 shows an exploded top-down view of the internal drive system. Referring now to FIG. 13, the interface between the drive gear 76 and the screw gear 94 can be observed. The screw gear 94 is fixed to the drive screw 92 and thus both are rotated when the motor (not shown) rotates the drive gear 76. The drive gear 76 is covered by the drive gear cover 96.

FIG. 14 shows the same exploded arrangement of components as FIG. 13 but from an alternative viewpoint. Referring now to FIG. 14, the wire port 98 and battery plug 100 can now be observed as part of the central housing 48.

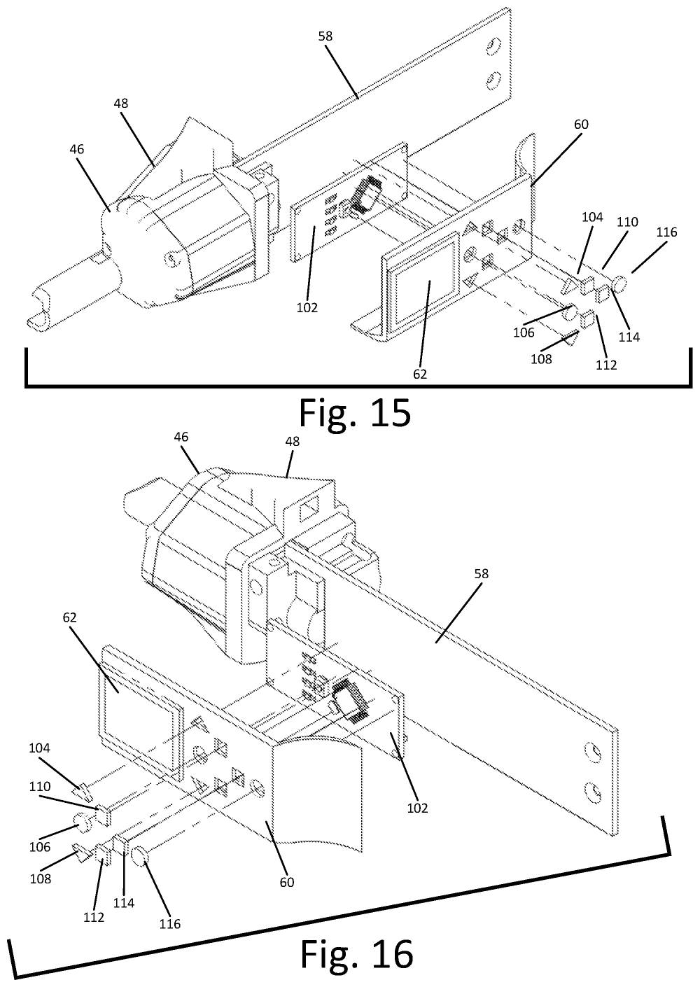

FIG. 15 shows an exploded top-front view of the electronic components of device 10 and internal to main body 45. Referring now to FIG. 15, an electronic control unit 102 is housed within a cavity between central column 58 and front panel 60 which receives signals from various actuators and buttons: index finger actuator (not shown), middle finger actuator (not shown), thumb actuator (not shown), up button 104, configuration button 106, down button 108, speed button 110, syringe button 112, reset button 114, and multiplier button 116.

When pressed, the configuration button 106 alters the mode of operation of device 10 between operation and configuration. While in operation mode, the up button 104 and down button 108 perform the same functions intended by thumb actuator (not shown) and middle finger actuator (not shown), which inject and aspirate, respectively. In configuration mode, the buttons adjust values relevant to the mode of operation, such as the ID. The syringe button 112, when used in configuration mode, allows the user to set a specific ID rather than a pre-identified ID based on common syringe manufacturers. The reset button 114 resets the values generated during operation and while in operation mode. The multiplier button 116 performs the same function as the thumb actuator (not shown) and sets the injection volume regardless of the current mode of operation. Similarly, the speed button 110 adjusts the motor (not shown) speed between a “slow” and “fast” setting regardless of the current mode of operation.

FIG. 16 shows the same exploded arrangement of components as FIG. 15 but from an alternative viewpoint. Referring now to FIG. 16, no new components are introduced and the view is provided for completeness.

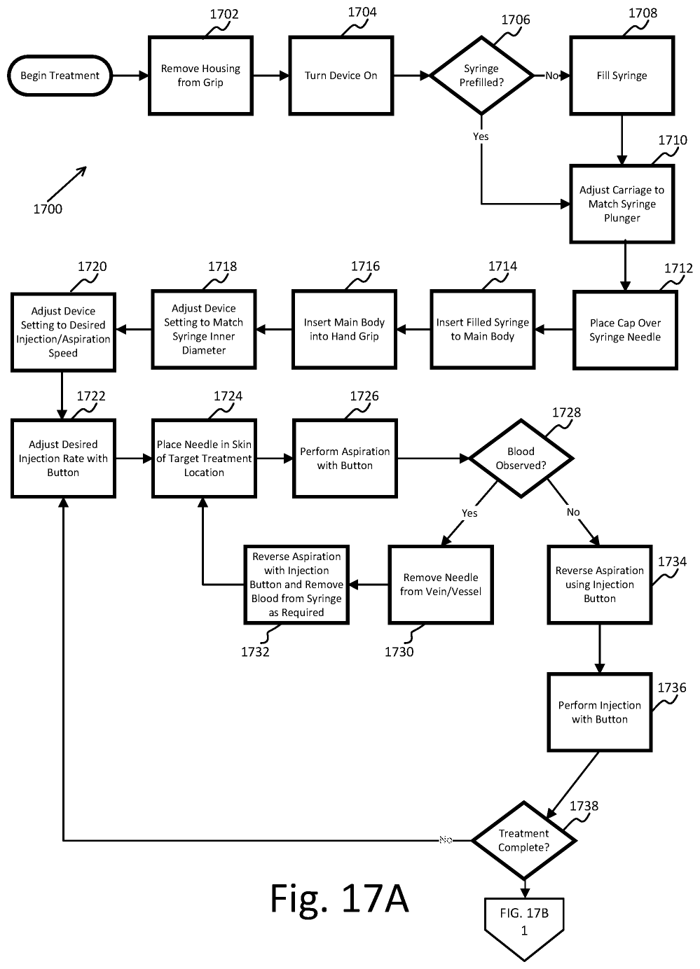

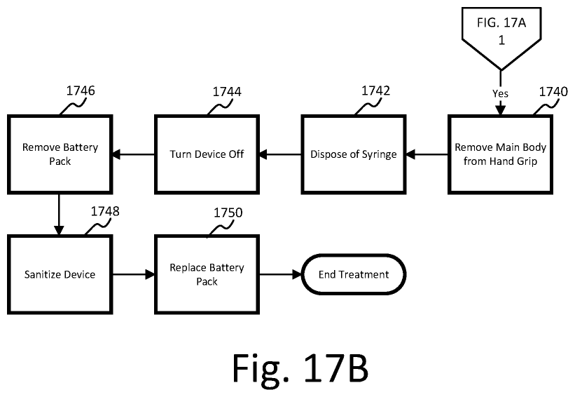

FIGS. 17A and 17B show the procedure of performing injections. Referring now to FIGS. 17A and 17B, the treatment procedure 1700 is defined. The procedure begins in block 1702 by removing the main body 45 from the hand grip 26 of device 10. In block 1704 device 10 is turned on. Block 1706 determines if the syringe 18 is prefilled. If yes, then control flows to block 1710. If the syringe 18 is not prefilled, then in block 1708 the syringe 18 is filled with the fluid required for the procedure at hand.

In block 1710 the plunger carriage 80 is adjusted to fit the length of the syringe 18. In block 1712 a cap is placed over the needle 22 of syringe 18 in order to prevent any contamination or damage. In block 1714 the syringe 18 is placed into the main body 45 syringe cavity 72 and the flange 74 of the syringe plunger 24 is inserted into the plunger carriage flange cavity 82 and the syringe body 20 is inserted into the syringe cavity 72. In block 1716 the main body 45 is inserted into the hand grip 26 to complete operational assembly of device 10.

As needed, in block 1718, device 10 can be adjusted to change the currently set ID. The ID will determine the base distance the motor 78 pushes the syringe plunger 24 with each injection with an intent to match a single unit of injection, commonly 0.01 milliliters. In block 1720 any desired speed settings are adjusted using the speed button 110. In block 1722 the primary loop of administering injections begins and allows for setting the injection rate with either the thumb actuator 42 or the multiplier button 116. In block 1724 the needle 22 is placed into a patient's skin at the site of injection. In block 1726, aspiration is performed using the middle finger actuator 40 or the down button 108. In block 1728, if blood is observed within syringe 18, then in block 1730 the needle 22 is removed from the vessel or vein and aspiration is reversed in block 1732 if a receptacle for blood removal is available. Control then returns to block 1724 to continue treatment. In some cases, aspiration may be reversed within the patient's skin and in others it may be reversed into some sort of receptacle to capture the blood safely depending on the available equipment, amount of blood and established procedures. Either method is acceptable and both are implied within the treatment procedure 1700.

In block 1728, if blood is not observed within syringe 18, then in block 1734 aspiration is reversed with the needle 22 still remaining in the same location in the patient's skin. Once aspiration is reversed, in block 1736 the currently loaded fluid is injected into the patient. At block 1738, a determination is made as to whether or not treatment is complete. If no, control is returned to block 1722 where injection rates are adjusted if needed and then control returns to block 1724 to repeat the cycle of injection. If the determination in block 1738 is yes, then in block 1740 (see FIG. 17B) the main body 45 is removed from the hand grip 26.

In block 1742 the syringe 18 is disposed using locally established procedures. In block 1744 device 10 is turned off. In block 1746 battery pack 52 is removed from main body 45. In block 1748 device 10 is sanitized using locally established procedures. In block 1750 battery pack 52 is replaced into main body 45 and treatment procedure is concluded.

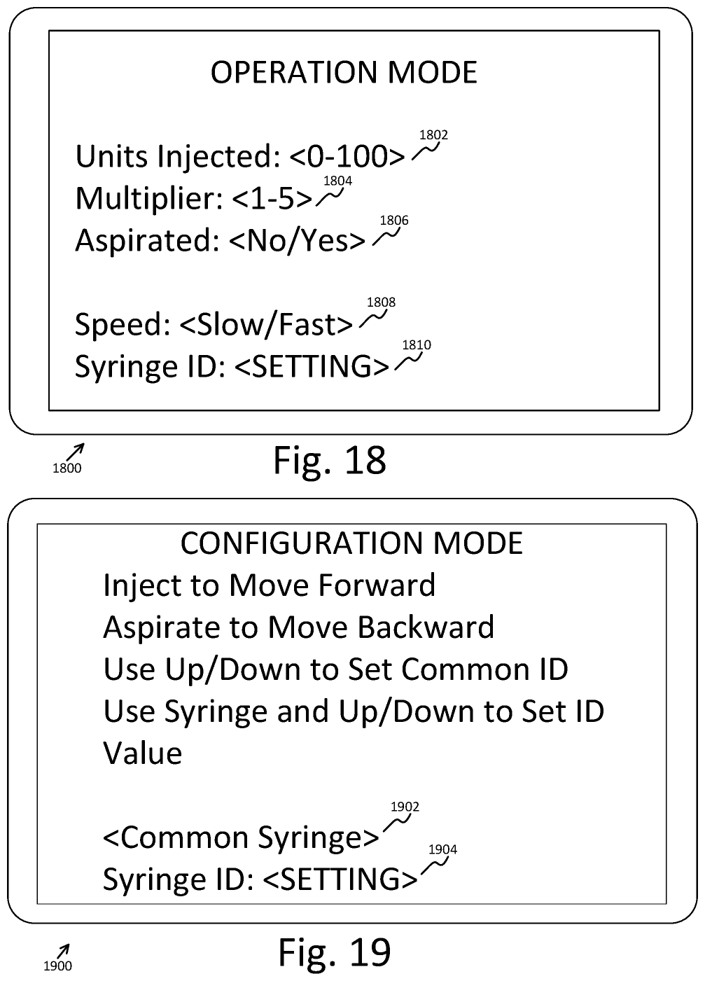

FIG. 18 shows the LED screen 62 with proposed key values relevant to using device 10 in operation. Referring now to FIG. 18, the screen 1800 titled “OPERATION MODE” is detailed with changing values. Operation mode is activated by pressing the configuration button 106 when in configuration mode. Otherwise, device 10 is already in operation mode. Value 1802 is an indicator to allow identifying the number of injection units already administered. Value 1802 is brought to 0 using the reset button 114 and is incremented by one unit times the currently set multiplier in value 1804 with each injection from either index finger actuator 38 or up button 104. Value 1804 is the currently configured multiplier value, adjusted by pressing thumb actuator 42 or multiplier button 116, which indicates the number of injection units injected per press of either index finger actuator 38 or up button 104. The maximum number of units is not expected to go beyond 100 in a single treatment. However, the maximum can be set to any number.

Value 1806 is indicative of whether or not the device 10 has currently been aspirated through the use of either middle finger actuator 40 or down button 108. When aspirated, indicated by “Yes” in value 1806, the next injection action will simply reverse the aspiration and indicate a “No” in value 1806. If aspiration has been activated multiple times since the last injection, each injection will reverse one aspiration and value 1806 will remain as “Yes” until all aspirations have been reversed.

Value 1808 indicates the currently set speed of motor 78 between “Slow” and “Fast” as the viscosity of the currently loaded fluid may necessitate a differing speed. A slower speed is generally associated with a higher viscosity of fluid. Finally, value 1810 indicates the currently configured ID of device 10. The ID will determine the base distance, not accounting for the multiplier value 1804, that the motor 78 pushes the syringe plunger 24 with each injection. The preferred measurement of injection volume is the “unit” or 0.01 milliliters. The goal of the ID value, dealing with cylindric volume calculations, is to ensure that each unit of injection as depicted by value 1802 is equal to 0.01 milliliters. By reverse-calculating the volume of 0.01 milliliters with a known cylindrical diameter, the motor distance can be altered by the electronic control unit 102 to maintain accuracy. If an injection site were to require more than one unit of injection, the multiplier value is manipulated with thumb actuator 42 or multiplier button 116 to adjust the number of units injected per press of the index finger actuator 38 or up button 104. Therefore, there is no specific value range required to constrain value 1810 but the ID of a common one milliliter syringe will be in the approximate range of 4.65 millimeters.

FIG. 19 shows the LED screen 62 with proposed key values relevant to using device 10 in configuration. Referring now to FIG. 19, the screen 1900 titled “CONFIGURATION MODE” is detailed with instructions and changing values. Configuration mode is activated by pressing the configuration button 106 when in operation mode. Otherwise, device 10 is already in configuration mode. The mode instructs that adjusting device 10 is possible in this mode in order to match the position of plunger carriage 80 to the length of the current syringe's plunger 24 prior to placing syringe 18 into main body 45 by using index finger actuator 38 and middle finger actuator 40. This is in contrast to the effect in OPERATION MODE indicated in FIG. 18 as the injection and aspiration buttons do not increment the value 1802. Value 1902 is a configured value that will set the device ID based on a named type of syringe. The range of possible values for value 1902 is reserved to the most common disposable-type syringes to speed the configuration process. Instructions are given on-screen to indicate how to change value 1902.

Alternatively, value 1904 can be used to specifically define the configured ID. Using the on-screen instructions, a raw value is set for value 1904 in place of a pre-configured value. Value 1904 will also change depending on the selected syringe given by value 1902. However, if value 1904 is manually changed then value 1902 will be blank.

Having described the present invention, it will be understood by those skilled in the art that many changes in construction and circuitry and widely differing embodiments and applications of the invention will suggest themselves without departing from the scope of the present invention.

What is claimed is:

1. A method for fine-precision injection for a treatment, the method comprising the steps of:
(a) placing a needle of an injection device into a skin at a first target site;
(b) performing an aspiration with the injection device;
(c) performing an injection with the injection device;
(d) placing the needle of the injection device into the skin at a second target site; and
(e) repeating steps (b) through (d) at a plurality of next target sites until the treatment is completed.
